(19) United States
(12) Patent Application Publication
(10) Pub. No.: US 2019/0244696 A1
(43) Pub. Date: Aug. 8, 2019

(54) MEDICAL RECORD MANAGEMENT SYSTEM WITH ANNOTATED PATIENT IMAGES FOR RAPID RETRIEVAL

(71) Applicant: Dharmendra Sushilkumar GHAI, Ulhasnagar (IN)

(72) Inventor: Dharmendra Sushilkumar GHAI, Ulhasnagar (IN)

(21) Appl. No.: 16/375,543

(22) Filed: Apr. 4, 2019

Related U.S. Application Data
(63) Continuation of application No. 15/946,512, filed on Apr. 5, 2018.
(60) Provisional application No. 62/645,540, filed on Mar. 20, 2018.

(30) Foreign Application Priority Data
Feb. 5, 2018 (IN) 201821004302

(51) Int. Cl. G16H 10/60; G16H 30/20; G06K 9/00

(52) U.S. Cl. CPC G16H 10/60; G06K 9/00087; G06K 9/00013; G16H 30/20

(57) ABSTRACT

A system and method for patient healthcare information management. The system includes a fingerprint scanner that generates fingerprint data by scanning the finger of a patient. That fingerprint data is forwarded to a hand scan server that performs a lookup to retrieve a corresponding patient ID or social security number. That patient ID or social security number is then sent to a healthcare server, such as at a hospital or other healthcare facility, to retrieve the healthcare information for the patient. That is particularly useful for patients who are unconscious or otherwise unable to recall or relay their identifying information and/or healthcare information history to the healthcare practitioner. In addition, the system can include an imaging capture device to take a picture of the patient. That image is displayed in conjunction with annotations that indicate the patient healthcare information history for the patient. That enables the healthcare practitioner to rapidly and easily see the patient's healthcare history and related details.

### RELATED APPLICATIONS

This application claims the benefit of U.S. Provisional Application No. 62/645,540, filed Mar. 20, 2018, and India Provisional Application No. 201821004302, filed Feb. 5, 2018, the entire contents of which are incorporated herein by reference.

### BACKGROUND OF THE INVENTION

### Field of the Invention

The present invention relates to providing patient healthcare information to healthcare workers. More particularly, the present invention manages patient healthcare information that includes annotated electronic images of patient healthcare issues.

### BACKGROUND OF THE RELATED ART

U.S. Pat. No. 8,744,147 describes a system that Electronic Medical Records (EMR) that includes images that can be annotated. U.S. Pat. Appl. Pub. No. 2009/0006131 describes an EMR system that includes past imaging information. U.S. Pat. Appl. Pub. No. 2014/0172457 teaches a medical information system that extracts predetermined information from collateral information and generates text information that is correlated with patient identification information.

When a severely wounded and/or unconscious patient is brought to an emergency room in a hospital, timely identification of the patient and the patient's medical history can be difficult. If the patient is unknown, treatment can be delayed since certain treatment parameters must be determined, such as identifying the patient's blood group. It is therefore important for healthcare workers to have immediate access to patient healthcare information, especially in a hospital emergency room.

In addition, current EMR system store a lot of information. The information may not be well organized and as a result it can take time for a heath care worker (e.g., physician, nurse, physician assistant, administrator) to locate the information they are looking for. For example, information about medical events (e.g., laboratory tests, x-rays) are typically arranged by date and the healthcare worker must sort through irrelevant information to find relevant information.

### SUMMARY OF THE INVENTION

Accordingly, a healthcare information system is needed that can be utilized by healthcare workers and healthcare facilities (such as hospitals, urgent care centers, physician offices, pharmacies) to manage healthcare information that helps address sorting through large amounts of data to identify relevant information. One object of the invention is to provide an electronic system that arranges medical event information on annotated electronic images of a patient, so that a user can quickly and reliably locate medical event information related to a current medical event or issue. A timeline of patient healthcare issues may be presented to quickly display medical information to the healthcare worker.

Thus, a system and method are provided for patient healthcare information management. The system includes a fingerprint scanner that generates fingerprint data by scanning a finger of a patient. That fingerprint data is forwarded to a hand scan server that performs a lookup to retrieve a corresponding patient ID or social security number. That patient ID or social security number is then sent to a healthcare server, such as at a hospital or other healthcare facility, to retrieve the healthcare information for the patient. Such a system may be useful for patients who are unconscious or otherwise unable to recall or relay identifying information and/or healthcare history information to the healthcare practitioner.

In certain embodiments, the system may include an imaging capture device to take a picture of the patient. That image may be displayed in conjunction with annotations that indicate the patient healthcare information history for the patient. This helps the healthcare practitioner to rapidly and easily see the patient's healthcare history and related details.

These and other objects of the invention, as well as many of the intended advantages thereof, will become more readily apparent when reference is made to the following description, taken in conjunction with the accompanying drawings.

### BRIEF DESCRIPTION OF THE FIGURES

FIG. 1 is a block diagram showing an overview of the system, in accordance with aspects of the present disclosure.

FIG. 2 is a block diagram of a portable device, in accordance with aspects of the present disclosure.

FIG. 3 is a block diagram of the operation of the system in accordance aspects of the present disclosure.

FIGS. 4 and 5 show Augmented ER screens, in accordance with aspects of the present disclosure.

FIG. 6 shows an Augmented ER screen having patient information and user-selectable options, in accordance with aspects of the present disclosure.

FIGS. 7A, 7B, and 7C show Augmented Imaging screens, in accordance with aspects of the present disclosure.

FIG. 8 shows an Encounter screen, in accordance with aspects of the present disclosure.

FIGS. 9A, 9B, and 9C show Augmented EMR screens, in accordance with aspects of the present disclosure.

### DETAILED DESCRIPTION OF THE PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in similar manner to accomplish a similar purpose. Several preferred embodiments of the invention are described for illustrative purposes, it being understood that the invention may be embodied in other forms not specifically shown in the drawings.

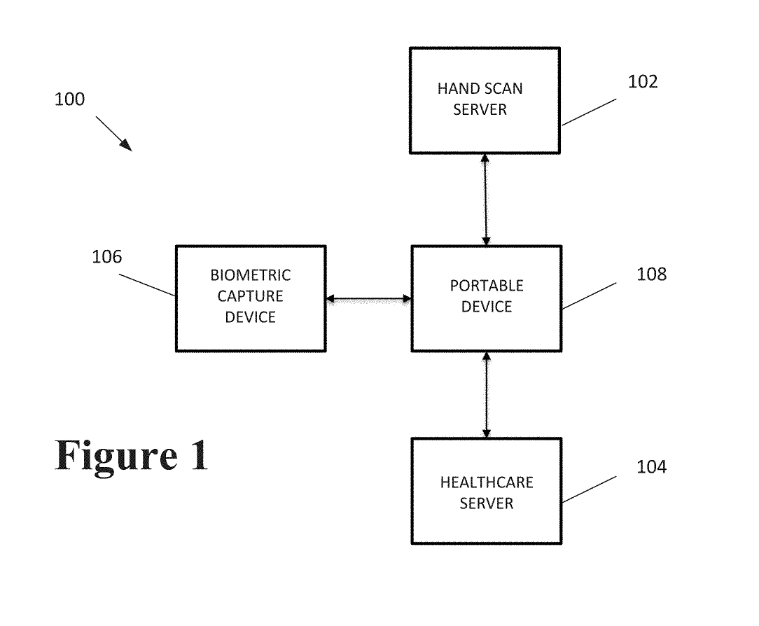

Turning to the drawings, FIG. 1 shows an EMR management system 100 in accordance with the invention. The system 100 includes a hand scan server 102, healthcare facility (e.g., hospital, urgent care center, etc.) or local server 104, and biometric capture device 106 such as a fingerprint scanner, and a portable device 108 operating one or more mobile applications, such as a smart phone or the like.

The portable device 108 may run an application that is hosted at a particular location, such as on the internet, or obtained from a store, such as an application store for download to the portable device 108. The external biometric device 106 may be used to obtain biometric information from the patient, which can be any biological data. In one embodiment, biometric information may be obtained from a patient's fingerprint. This device can be integrated with the portable device 108, such as by touching the patient's finger to the touchscreen of or a sensor positioned on the portable device 108. In certain cases, the biometric capture device 106 can be connected to the portable device 108 via a USB port, wirelessly, or other connection capable of connecting a peripheral device to another device, to transfer the captured biometric information to the application. The biometric capture device 106 can, for example, scan the patient's finger to obtain fingerprint data in accordance with any suitable technique, such as for example to obtain an electronic representation of the fingerprint, in any supported format, for comparison against a set of known fingerprints. While the discussed in conjunction with an embodiment which utilizes fingerprints for biometric information, other biometric information may be used, such as iris recognition, facial recognition, voice or speech patterns, genetic markers, etc.

In one embodiment of the invention, the hand scan server 102 is at a central location and can be accessed by one or more facilities, locations, or portable processing devices 200. The hand scan server 102 can include or can communicate with one or more storage devices to store patient biometric information, such as fingerprint information (collectively referred to below as just “fingerprint information”), of patients. The stored fingerprint information may be regularly updated with fingerprint data for patients. For example, fingerprint data associated with patients new to the system, including newborns, nay be added. A unique patient ID or Patient Access Number may be stored in association with each patient fingerprint data stored. Additional patient identification or information may also be stored, as needed. The patient ID may, in certain cases, be generated by a hospital information system (HIS) operating as a part of the healthcare facility server 104.

The patient ID and associated patient biometric information (i.e., fingerprint data) can be obtained in any suitable manner. For example the HIS 212 can create a patient ID and associate that patient ID with the patient's fingerprint data, both preexisting, or obtained during a check-in procedure, for existing and new patients. That information can then be transmitted to the hand scan server 102 from time to time or as the information is updated. In certain cases, the hand scan server 102 can obtain that information from a plurality of HIS from various respective hospital servers 104, and cross-reference the information, for example, based on biometric information or an external reference identifier, such as a social security number. Where various healthcare servers 104 generate a different patient ID for the same patient, those different patient IDs can be stored by the hand scan server 102 in association with the patient biometric information. The portable device 108 communicates with the hand scan server 102, for example through a computer network or direct connection, using, for example, web services operated by or in communications with the server. Examples of computer networks include the internet, intranets, cellular networks, WiFi, or any other suitable computer network.

The healthcare facility server 104 may be maintained by a local administrator, such as a hospital IT team. The healthcare facility server 104 may include a storage device that stores the medical history of the patient, for example, in Health Level-7 (HL7) data format, which is a standard for transfer of data between various healthcare providers.

In certain embodiments each healthcare facility can have its own healthcare facility server 104, and the healthcare facility servers 104 can be in communication with each other via one or more computer networks. In other embodiments, a single centralized healthcare facility server 104 can be provided that communicates with healthcare computers located at healthcare facilities. In other embodiments, the hand scan server 102 can be provided at one or more of the healthcare facility servers 104. In yet another embodiment, a mobile application on the portable device 108 sends a request to the healthcare facility server 104 and the healthcare facility server 104 returns the requested data from that healthcare facility server 104 or from data consolidated from amongst multiple healthcare facility servers 104, to the portable device 108.

In operation, a mobile application on the portable device 108 receives biometric data (e.g., fingerprint data) from the biometric capture device 106, then transmits that data to the hand scan server 102. The hand scan server 102 retrieves the patient ID from its associated storage device based on the biometric data, and sends the patient ID to the mobile application on the portable device 108. The mobile application on the portable device 108 can then send the patient ID to the healthcare facility server 104. In response, the healthcare facility server 104 retrieves the patient's EMR data from its database, and transmits that data to the mobile application on the portable device 108. According to certain aspects, this data may be in a HL7 data format. By centralizing stored fingerprint data at the central hand scan server 102, a greater database of fingerprint data can be accumulated and provided to the mobile application on the portable device 108.

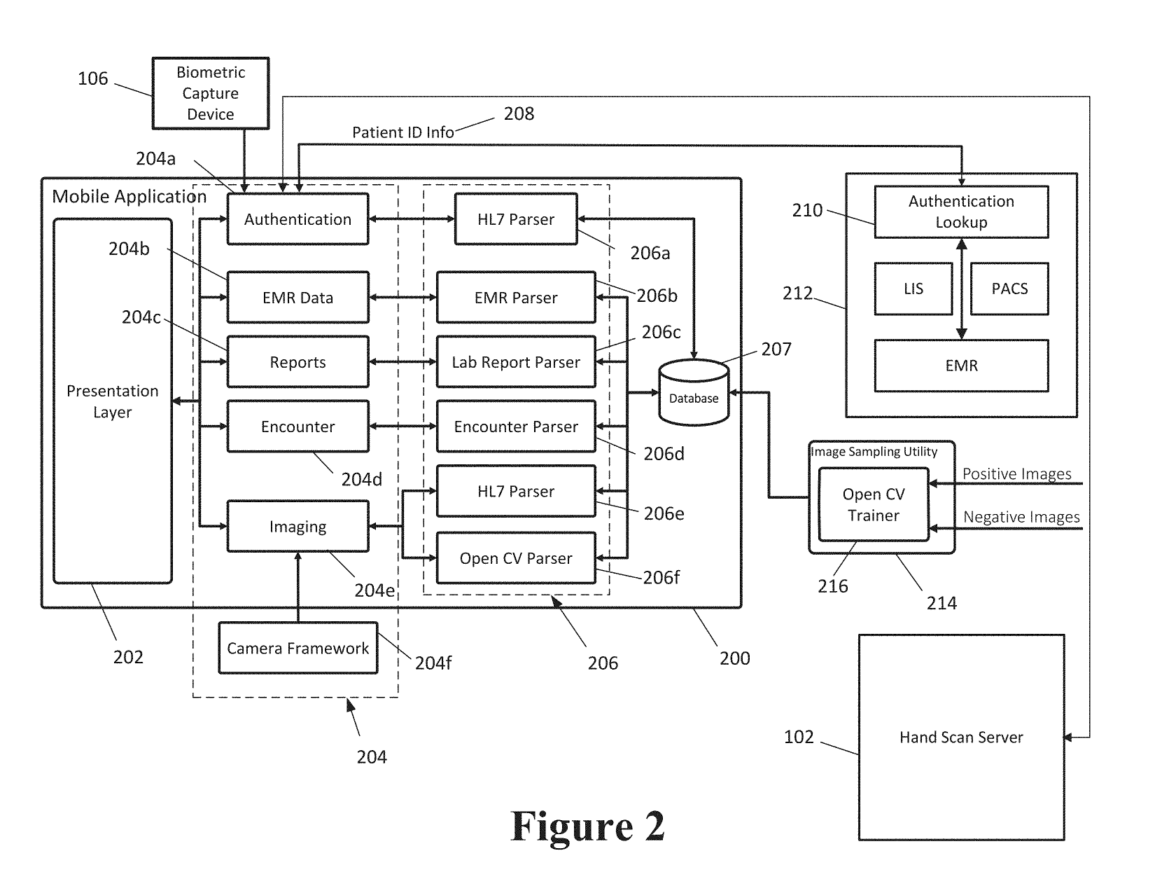

FIG. 2 is a block diagram of an EMR management system, in accordance with aspects of the present disclosure. The mobile application 200 includes a presentation layer 202, one or more operation modules 204 and one or more data parsers 206. More specifically, examples of operational modules 204 include an Authentication Module 204(a), EMR Data Module 204(b), Reports Module 204(c), Encounter Module 204(d), Imaging Module 204(e), and Camera Framework Module 204(f). According to certain aspects, operation modules may be located external to the mobile application 200, such as with the Camera Frame Module 204(f), and connected to the mobile application 200, for example by a USB cable and interface). The data parsers 206 include, for example, an HL7 Parser 206(a), EMR Parser 206(b), Lab Report Parser 206(c), Encounter Parser 206(d), HL7 Parser 206(e), and Open CV Parser 206(f).

The mobile application 200 also includes a storage 207 such as a database, and a presentation layer 202. The storage 207 can be in communication with the parsers 206. The presentation layer 202 can be in communication with the operational modules 204. In general, the parsers 206 retrieve information from the database 207, and prepare or parse the data into a format for use by the operational modules 204. The operational modules 204 process the parsed data and this parsed data may be displayed on a display screen of the mobile application 200 by the presentation layer 202. The presentation layer 202, operational modules 204, and parsers 206 can be run or executed by a processing device of a portable device.

According to certain aspects, the mobile application 200 may obtain an identity of a patient either through an assigned identifier, such as a patient ID number, or via biometric information. The authentication module 204(a) operates to help identify and authenticate a patient. Where biometric information is used, the authentication module 204(a) interfaces with the biometric capture device 106, such as a fingerprint scanner. In this example, the authentication module 204(a) receive fingerprint data from a scanned finger from the biometric capture device 106. The authentication module 204(a) then transmits the received fingerprint data to the hand scan server 102. The hand scan server 102 compares the fingerprint data with fingerprint data for a set of patients stored at the hand scan server 102. If there is a match, the hand scan server 102 retrieves the associated patient identification information (e.g., patent ID or other information that identifies a patient) and transmits the patient identification information back to the authentication module 204(a).

If there is not a match between the fingerprint data as against the fingerprint data for a set of patients stored at the hand scan server 102, the authentication module 204(a) may then passes the fingerprint data to an authentication lookup module 210 of the HIS 212. While the authentication lookup module 210 is shown in this this example as incorporated into the HIS 212, the authentication lookup module may be provided separately from the HIS 212, for example as a stand-alone server, online service, or as a part of another service. The authentication lookup module 210 may then compare the fingerprint data against fingerprint data for a set of patients stored at the HIS 212, for example as a part of EMR. If there is a match the patient is identified, the authentication lookup module 210 may retrieve the associated patient identification information and transmit the patient identification information back to the authentication module 204(a). If there is not a match between the fingerprint data and data stored in the HIS 212, then another option to identify the user may be presented, such as directly entering an assigned identifier to the mobile application 200.

Where the assigned identifier, such as a patient ID number is provided to the mobile application 200, the received assigned identifier may be passed to the authentication lookup module 210 of the HIS 212. The authentication lookup module 210 may then search the HIS 212 records for a matching patient ID and if there is a match, the patient is identified.

The authentication module 204(a) may also receive, from the authentication lookup module 210, medical data associated with the patient identified by the patient identification information. In this example, the authentication lookup module 210 requests patient medical information from the EMR module. The patient medical information may include, for example, all historical and current medical records for the patient available, or a subset of a patient's medical records. The patient medical information may, for example, be stored in a HL7 format. The patient medical information may be received by the authentication module 204(a) and passed to the HL7 Parser 206(a).

The parsers 206 generally organize bulk data received from the HIS 212 into a format useable by the presentation layer 202, which helps ensure a smooth transfer of data from the operational data modules 204 to the presentation layer 202 when requested by the presentation layer 202. Patient medical information received from the authentication module 204(a) may be parsed by the HL7 Parser 206(a) to segregate the data into EMR data, Lab Report data, and Encounters data. Generally, patient medical information will include different types of medical information related to the patient's medical history (e.g. past treatments, allergies, notes, observations, etc.), lab reports, and imaging data (e.g., X-ray, computed tomography (CT) scans, magnetic resonance imaging (MRI), etc.). Segregating this data may allow improved processing as not every type of medical information may need to be displayed at once and performance may be increased by not requiring parsing all of a patient's medical information when just a single type of medical information is needed. This segregated data may be stored in a database 207.

The EMR parser 206(b) is used to organize the patient's medical history, such as allergies, medications and past treatments, in a suitable way to be displayed at the presentation layer. These details may be displayed based on the body part selected. The lab report parser 206(c) is used to organize the lab reports of the patient received from the HIS 212 in a suitable format to be displayed at the presentation layer 202. The encounter parser 206(d) structures possible multiple consultations of a patient with one or more physicians, containing, for example, details related to a physician visit, such as appointment date/time, consult date/time, name of physician, department, etc. The OpenCV Parser 206(f) receives each frame being taken by camera framework and compares it with the output from an Open CV Trainer 216 to identify if a body part of interest has been captured by camera.

Data associated with different types of medical information may be provided independently. For example, the presentation layer 202 may allow users to specifically request particular types of data. Where a request for medication information is received by the EMR data module 204(b) from the presentation layer 202, the EMR data module 204(b) requests the medication information from EMR parser 206(b). The EMR parser 206(b) may then access the database 207 to retrieve and parse EMR data to obtain the medication information. This medication information may be formatted for display and then returned to the EMR data module 204(b) for display by the presentation layer. In certain embodiments, parameters may be provided to return EMR data that are within the parameters. For example, one or more dates may be provided as a parameter along with the requested type of EMR data, such as medication information. The type of EMR data that satisfies the one or more parameters may then be returned to the EMR data module (204b), such as medication data that is before, after, or between the provided dates.

Where details regarding lab reports, such as for lab tests performed and lab results, are requested, the reports module 204(c) may request lab reports from the lab reports parser 206(c). The lab reports parser 206(c) may then access the database 207 to retrieve, parse, and format lab report data for return to the reports module 204(c) and display by the presentation layer 202. Parameters may also be provided to help specify which lab reports, tests, dates, etc. to retrieve.

Where details regarding a patient's consultation history are requested, the encounter module 204(d) may request such information from the encounter parser 206(d). The encounter parser 206(d) may retrieve such information from the database, parse, format, and return the data to the encounter module 204(d) for display by the presentation layer 202. Parameters, such as dates, times, specific physicians, etc. may be provided.

The imaging module 204(e), together with the open CV parser 206(f), processes the image frames received from the camera framework 204(f) and marks the body part in interest if available in the frame. The camera framework module 204(f) captures video of the patient and passes images frames to the OpenCV parser 206(f) to detect whether body parts of interest are available within the frame. According to certain aspects, the OpenCV parser 206(f) may execute a machine learning model for detecting various body parts. For example, the OpenCV parser 206(f) may include a set of classifiers for characteristics of an image. The OpenCV parser 206(f) may receive a machine learning model including associated weights for these classifiers for configuring the classifiers to recognize various body parts. Where a specific body part is designated as one of interest, if the body part of interest is available within the frame, then the frame is marked with an icon overlaid in the presentation layer 202. In addition, where details related to images or scans, such as X-ray, MRI and CT scans, of a patient are requested, the imaging module 204(e), along with the HL7 parser 206(e), displays imaging data received from the HIS 212.

According to certain aspects, the HIS 212 may include a Lab Information System (LIS), the Electronic Medical Records (EMR), and the Picture Archiving and Communication System (PACS). The LIS stores the lab reports, the EMR stores the medical history of the patient, and the PACS stores the images like MRI and CT Scan.

The OpenCV trainer module 216 of the image sampling utility 214 may be used to train one or more machine learning models for use by one or more parsers 206, of the mobile application 200 during a training phase. Generally, this training phase is performed remotely from the mobile application 200 and the one or more machine learning models may be stored/updated in storage 207 during, for example, a software update or during initial configuration of the mobile application 200. According to certain aspects, OpenCV parser 206(f) utilizes a machine learning body parts model to help identify the body part in each image frame provided by the camera. This model may be provided by the OpenCV trainer module 216. The OpenCV Trainer 216 trains the machine learning body parts model utilizing a predetermined set of positive and negative body part images for training. The database 207 stores the data obtained from the HIS 212, such as EMR data, lab reports and encounters along with basic patient details like age and gender.

The authentication module 204(a) of the mobile application 200 receives authentication information from a user, such as the patient's ID or fingerprint. The authentication module 204(a) uses that authentication information to accesses patient medical information stored in the healthcare server 104, such as patient medical data stored on the HIS 212. When fingerprint data is received by the authentication module, the fingerprint data may be used to retrieve the corresponding patient ID from the hand scan server 102. If the patient ID is provided to the authentication module 204(a), then the patient ID may be sent to the healthcare facility server 104 to obtain the patient medical information from the healthcare facility server 104. The authentication lookup module 210 may be maintained along with or a part of the hospital/healthcare servers. This network topology helps prevent malware attacks. This authentication lookup module 210 can identify the authorized requests and pass those requests to the HIS 212 or block the unauthorized requests and respond from the module itself.

The image sampling utility 214 constructs a machine learning body parts model that may be used by the mobile application 200 to detect images of various body parts. The utility 214 receives and stores a set of body part images, for example about 1000 images of hands with different textures and in different positions as positive images along with images without a hand as negative images. A machine learning model may include a set of weights used by a set of classifiers which are trained to recognize characteristics of an input, such as an image. During training, classifier may be adjusted based on the training images and whether a particular training image contains the body part in question. The resulting model for a particular body part may be stored in, for example, a data file, such as an XML file. Similarly separate files may be generated for each body part. These files are used by the Open CV parser 206(f) to identify the body part in an image frame provided by the camera framework.

As shown in FIG. 2 and discussed above, the operational modules 204 and parsers 206 are located at the mobile application 200. However, in an alternative embodiment of the invention, an intermediary processing device can be provided to pre-process data for transmission to the mobile application 200. That would make it easier for the mobile application 200 since it would then receive information at the presentation layer 202. The pre-processing can occur at a processing device that is in communication with the mobile application 200 and/or healthcare server 104, such as for example the hand scan server 102, or another separate server accessible directly or via a network or internet.

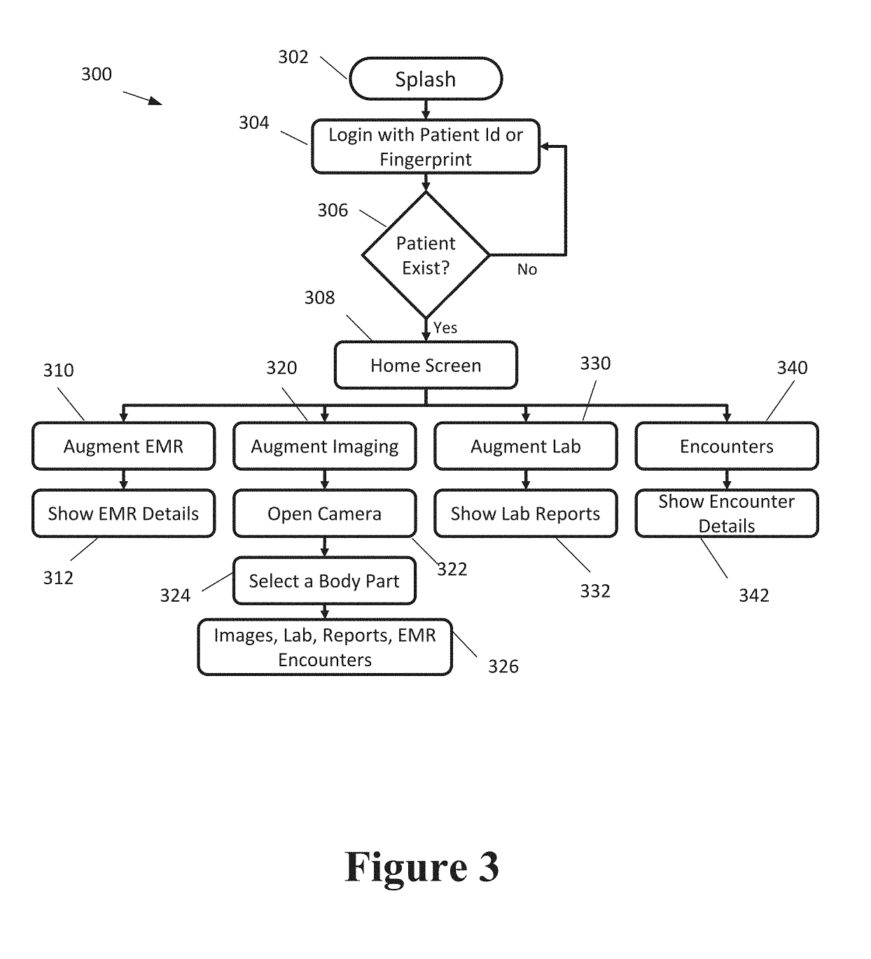

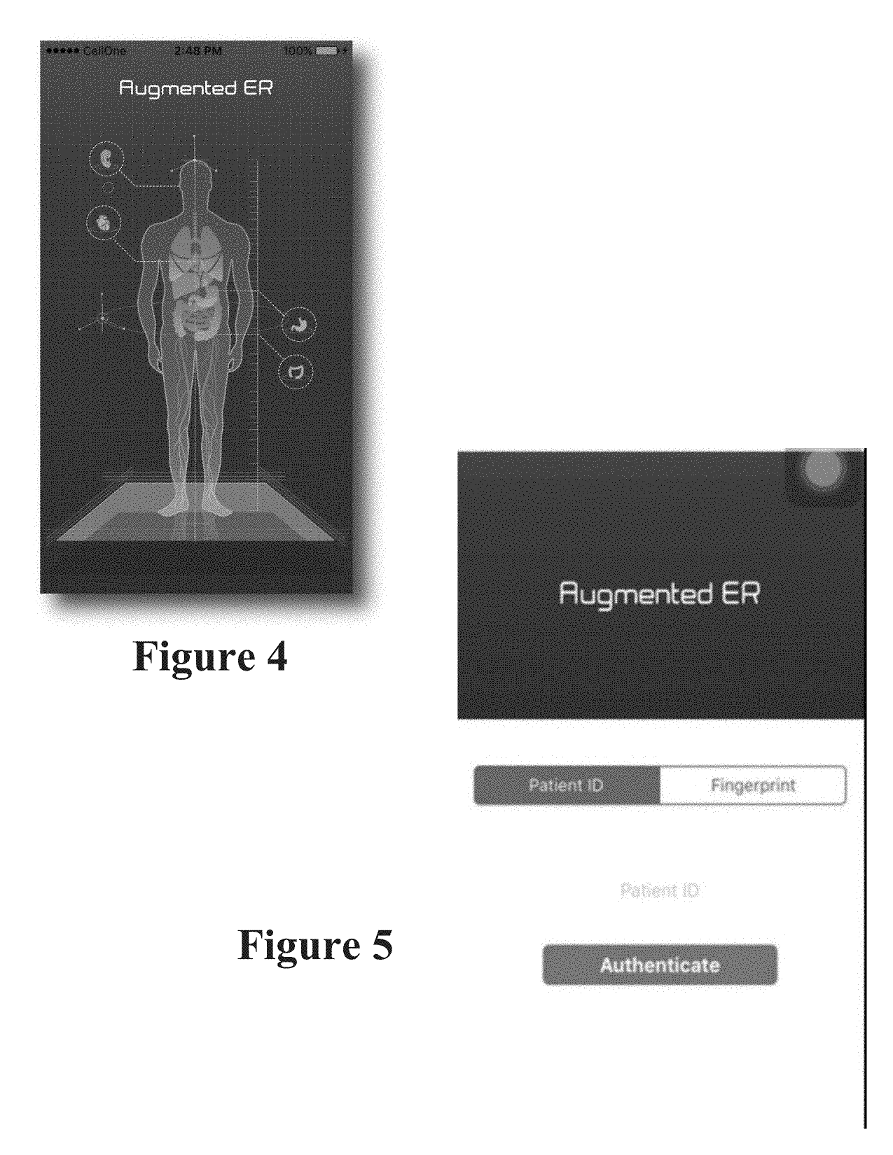

Turning to FIG. 3, the operation 300 of the system 100 will be discussed. The mobile application 200 may be operated by a healthcare user, such as a physician, nurse, physician's assistant, laboratory technician, and/or hospital room staff. The mobile application 200 starts with a splash screen (FIG. 4), step 302, followed by an authentication screen (FIG. 5), which are displayed on the mobile application 200. At step 304, to log in on the authentication screen, the healthcare user or the patient, enters patient identification information into the mobile application 200. The patient identification information can be, for example, patient ID, or patient biometric information such as a fingerprint. The user can select the type of patient identification information that will be entered on the authentication screen, as shown in FIG. 5. If a fingerprint (or other physical attribute or biometric, such as a retinal scan) is selected, the patient's finger may be placed on a fingerprint sensor, which scans the patient's fingerprint. The fingerprint sensor, or other biometric capture device, may be a separate device that is connected, either wired or wirelessly, to the mobile application 200, for example, via a USB, Bluetooth, or other such connection.

The authentication operation is handled by the authentication module 204(a) (FIG. 2) of the mobile application 200. To help support authentication using biometric information, the authentication module 204(a) may obtain biometric information from the healthcare facility server 106. The authentication module 204(a) may then send this biometric information to the hand scan server 102. The biometric information may be associated with patient identification information and stored on the hand scan server. The hand scan server 102 may receive a request to match a particular biometric, such as a fingerprint from the mobile application 200. If a match is found, the hand scan server may send the identification information to the authentication module 204(a) of the mobile application 200. The authentication module 204(a) may then send the patient identification information to the healthcare facility server 104 to retrieve patient details, such as medical records. In the case where the patient identification information 208 is provided to the authentication module 204(a), the patient identification information will be sent directly to the healthcare facility server 104 to retrieve the corresponding medical details.

At step 306, the mobile application 200 transmits the scanned fingerprint to the hand scan server 102 to attempt to retrieve patient identification information. The hand scan server 102 looks up the fingerprint to find and retrieve the corresponding patient identification information. More specifically, the authentication module 204(a) attempts to obtain the patient identification information corresponding to a fingerprint from the scan server 102, if it is available. The patient identification information is then passed to the authentication lookup module 210. If the authentication lookup module 210 responds with patient details (i.e., by sending the patient healthcare history data to the mobile application 200), the patient identification information exists in the HIS and the received patient details are associated with the patient identification information.

If the hand scan server 102 does not recognize the biometric data, the system remains at the authentication screen (FIG. 5). If the biometric data is recognized, the hand scan server 102 sends the patient identification information to the mobile application 200. The mobile application 200 can then transmit the patient identification information to the healthcare facility server 104. The healthcare facility server 104 stores patient medical data associated with patient identification information. The healthcare facility server 104 receives the patient identification information from the mobile application 200 and sends the patient medical data (also referred to herein as patient healthcare history data) to the mobile application 200. Thus, the healthcare user is able to obtain the patient identifying information and medical records even if the patient is unconscious or unable to speak coherently. The medical records can include basic details of the patient, such as for example name, age, gender and address, the treatment details (e.g., the time of treatment), the images of X-Ray or URL links to get the images. It also includes the details like medication, allergies of the patient.

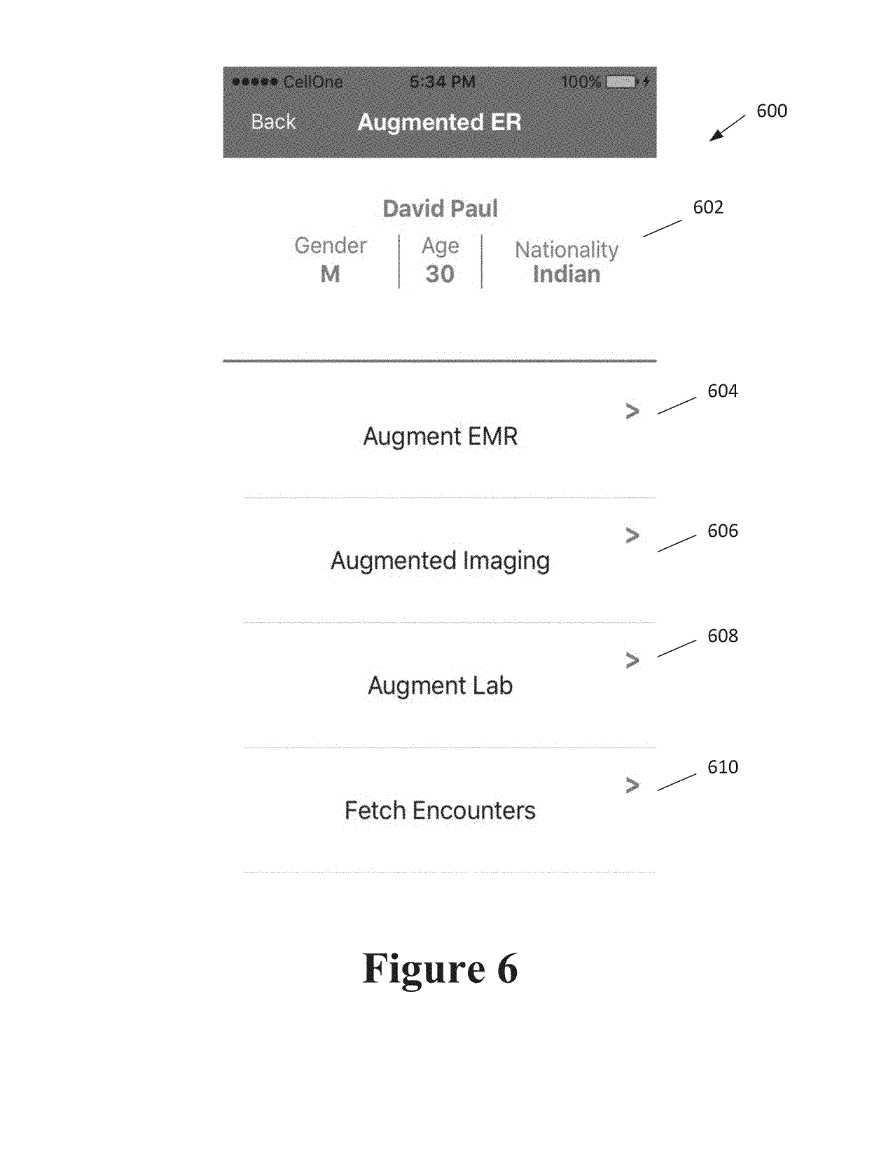

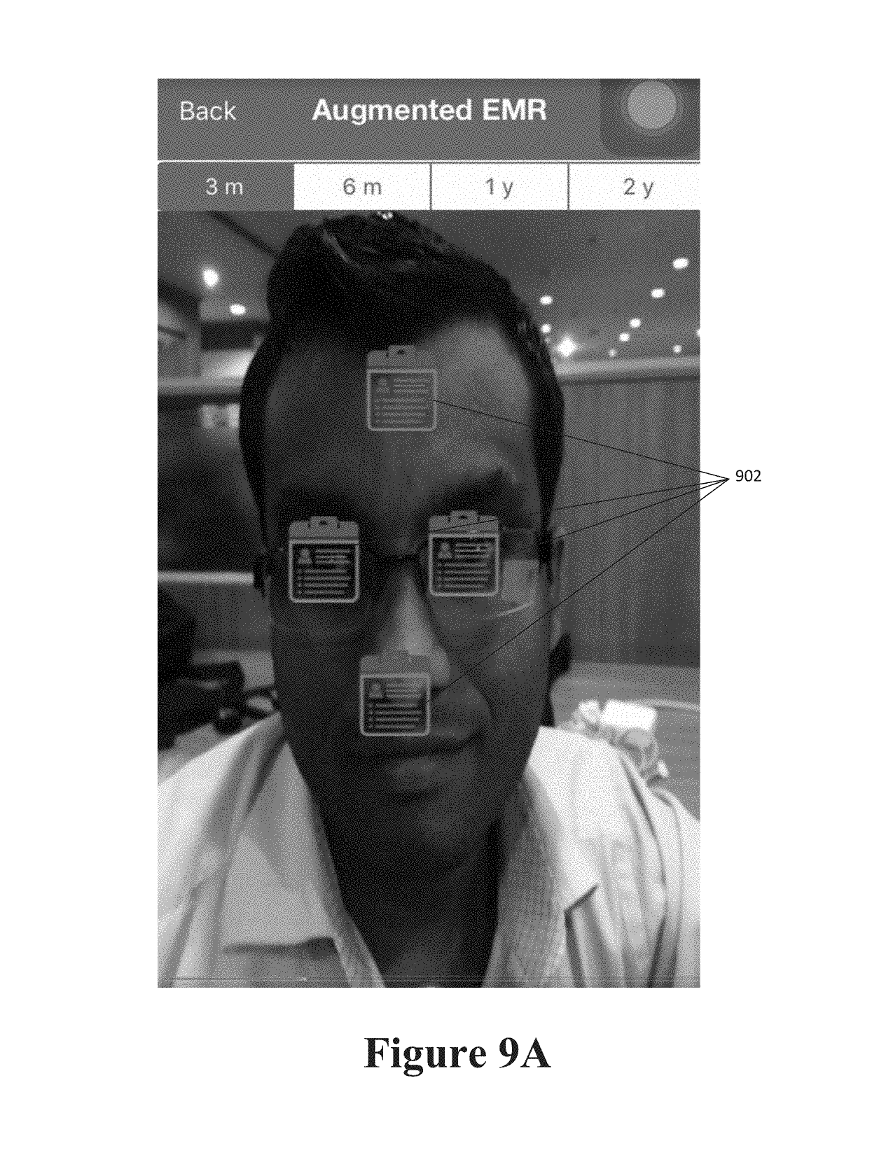

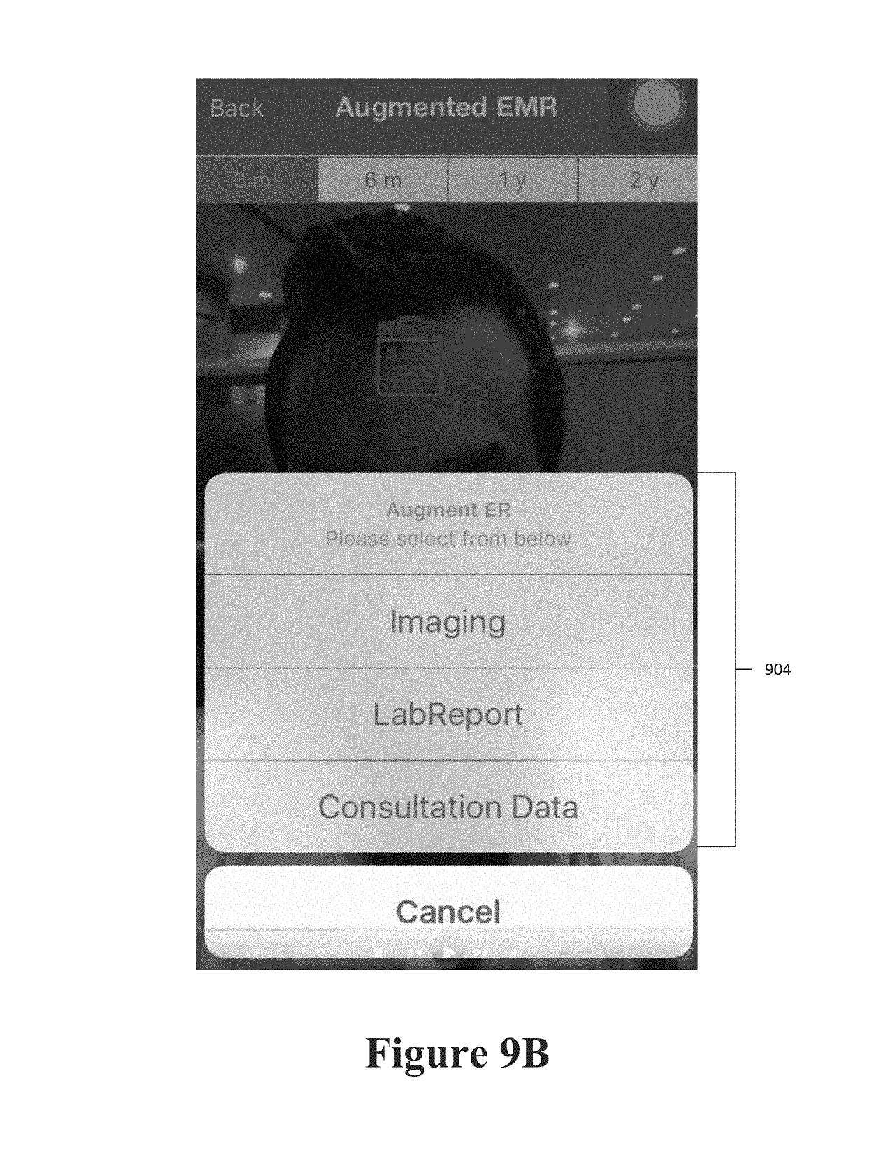

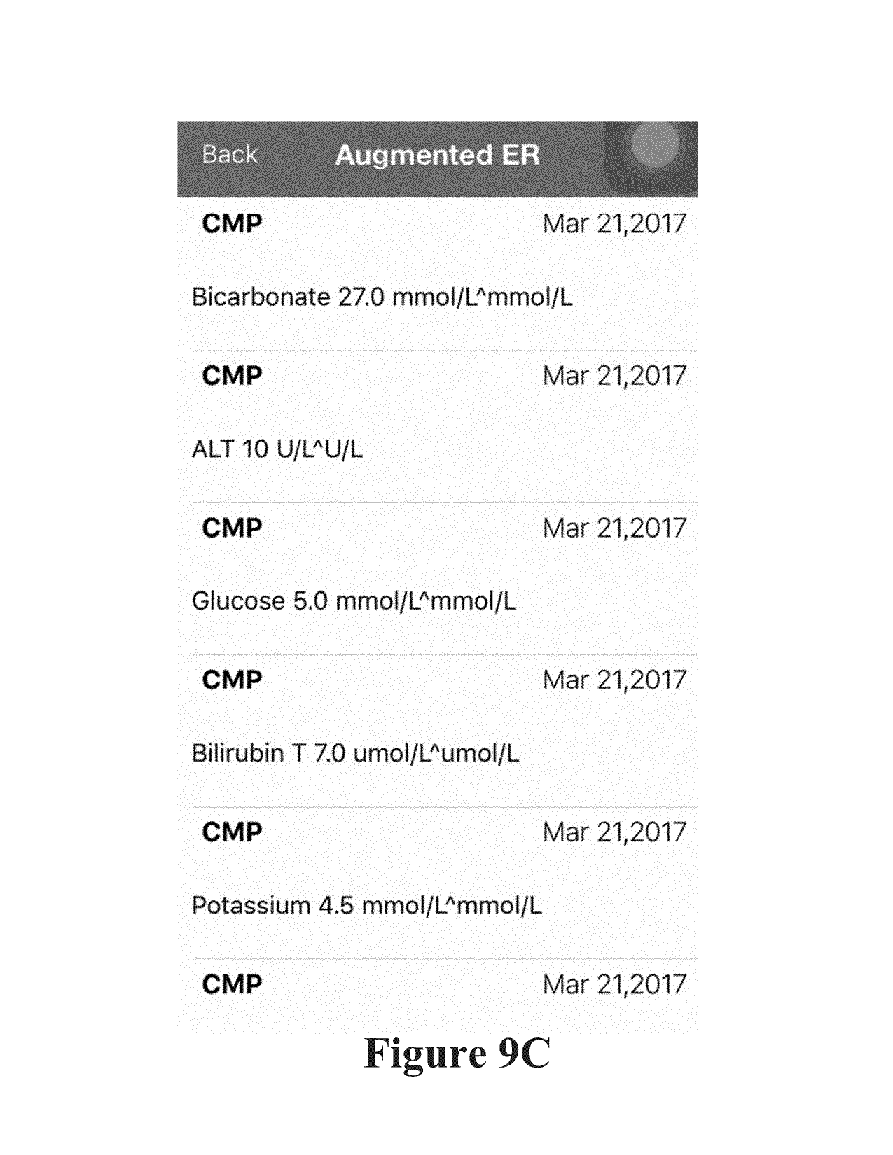

Once the patient is authenticated, a home screen (FIG. 6) is displayed on the mobile application 200, step 308. The home screen includes a summarization of patient information 602, such as the patient's name, gender, age and nationality. The home screen also includes operation selections that are available to the healthcare user, such as: Augment EMR 604, Augmented Imaging 606, Augment Lab 608, and Fetch Encounters 610. The healthcare user can select any one of those operations 604-610, for example, by clicking on the text or other UI element.

Where the Augment EMR 604 operation is selected from the Home Screen, the user is presented with the Augmented EMR screen, at step 310 and as shown in FIG. 9A. At step 322, the camera connected to the mobile application 200 is activated. The healthcare user may then point the camera at the patient to capture a live video stream of the patient. As shown, the video image is displayed by the mobile application 200. Icons 902 may be overlaid on the image or video stream. These icons 902 may be positioned on portions of the patient associated with a past medical history. For example, icons 902 are displayed overlaying the user's forehead, nose, and both eyes. The icons 902 may be overlaid based on information from the patient's medical history. For example, EMR records may be parsed to determine body part locations noted in the EMR records. These body part locations may be used to designate body parts of interest and the image or video captured by the camera may be parsed, as discussed in conjunction with the OpenCV parser 206(f) to identify those body parts of interest. Icons 902 may then be overlaid on the identified body parts of interest, here the patient's forehead, nose, and both eyes.

The healthcare user can then select one of the icons 902 from the display and a menu 904 of related options may be displayed. The menu 904 may be based on the medical records associated with a particular selected body part. Here, the user then has the option to see various medical information for the patient, such as Imaging, Lab Reports, and Consultation Data. If the user selects lab report from FIG. 9B, the mobile application 200 retrieves that data and displays it at FIG. 9C. For example, in FIG. 9C, the displayed medical history can provide results for a Comprehensive Metabolic Panel (CMP), which is a panel of 14 blood tests that serves as an initial broad medical screening tool.

It is noted that the information displayed at FIG. 9C can also optionally be accessed by the user selecting “Augment Lab” (which can also be called “Diagnostic Reports” or the like) 608 from screen FIG. 6. However, that will provide all reports for the patient, and not just those limited to a specific location of the patient.

Where the Augment EMR 604 operation is selected by a user from the home screen 600 EMR data from the healthcare facility server 104 is displayed by the mobile application 200 at step 312. The Augment EMR 604 operation is provides a data summary of the patient, and may be utilized to display the medical history of a patient. For example as shown, the mobile application 200 displays a summary of patient identification information 602, such as the patient's name, gender, and age. Upon selecting Augment EMR 604, the user is presented with FIG. 9A. The user can then select an icon of icons 902 overlaid on body parts visible in the image to obtain detailed information about that body part for the patient.

The Augment EMR operation 604 may be performed by the EMR Data module 204(b) of FIG. 2). Patient medical records may be received from the HIS 212 and parsed by the HL7 Parser 206(a) into segregated data portions including EMR data, lab report data, and encounters data and stores these segregated data portions into database 207. The EMR parser 206(b) may access, for example, the EMR data stored in database 207 and parse the EMR data to organize the EMR data for the EMR Data module 204(b). For example, EMR data may be divided into multiple segments. Segments may contain information generally related to a specific event, such as an admission, procedure, discharge, etc. Generally, segments contain one or more fields of data encoded in a standardized format, such as HL7. These fields may be parsed by the EMR parser 206(b) to categorize the data in various ways. For example, here, the EMR parser 206(b) categorizes the EMR data based on the body part affected. In this example, the data related to the patient's eye is associated together and the data related to other body parts are also respectively associated together.

The camera framework 204(f) captures video frames, sending them to the OpenCV parser 206(f). Based on the machine learning body parts model of the OpenCV parser 206(f), the particular image frame is analyzed for body parts of the patient visible in the particular image frame. If the body part is detected the image frame is sent to the presentation layer 202 along with coordinate position of the detected body part. The presentation layer 202 may then annotates the image frame to overlay, for example, icons and information, for display to the user. Different icons may be displayed based on the type of information represented. For example, the OpenCV parser 206(f) may also categorize dates associated with events and vary an icon size, shape, color, type, etc. based on how recently an event occurred. Once an icon is selected by a user, a view appears as in FIG. 9B. After selecting the required information, the application identifies the body part for the selected icon and displays the information for the selected body part.

If the LabReport operation is selected from menu 904 of FIG. 9B, the presentation layer 202 sends a request to the EMR data module 204(b). The EMR data module 204(b) may access data parsed and categorized by the EMR Parser 206(b) appropriate for display based on the request. Based on the request received from the presentation layer 202, the EMR parser 204(b) may categorize EMR data stored in the database 207 based on the request and replies with information on LabReports for the request body part. The presentation layer 202 may then displays the screen as shown in FIG. 9C with Lab Reports for the selected body part. Similar operations may be performed for other available options, such as Augmented Imaging 606, Augment Lab 608, and Fetch Encounters 610, although the exact workflow may be altered as discussed below.

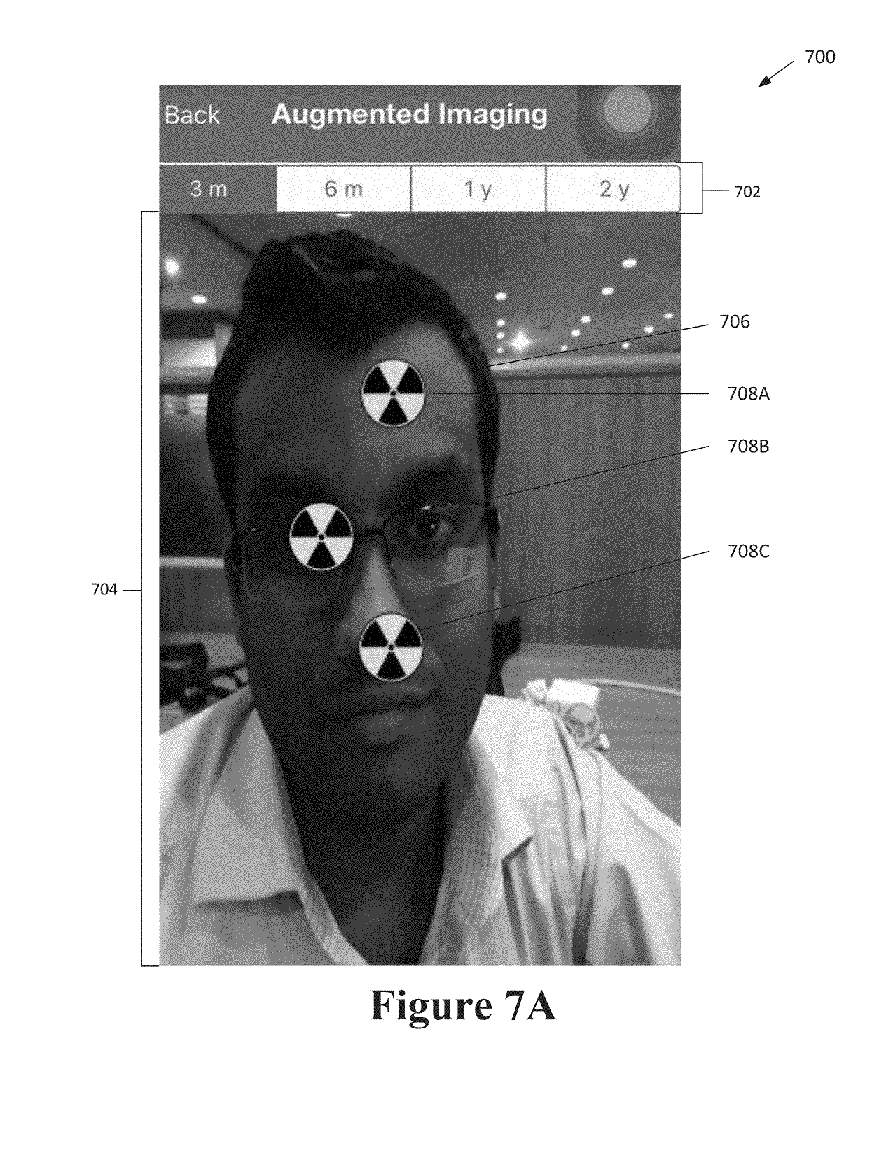

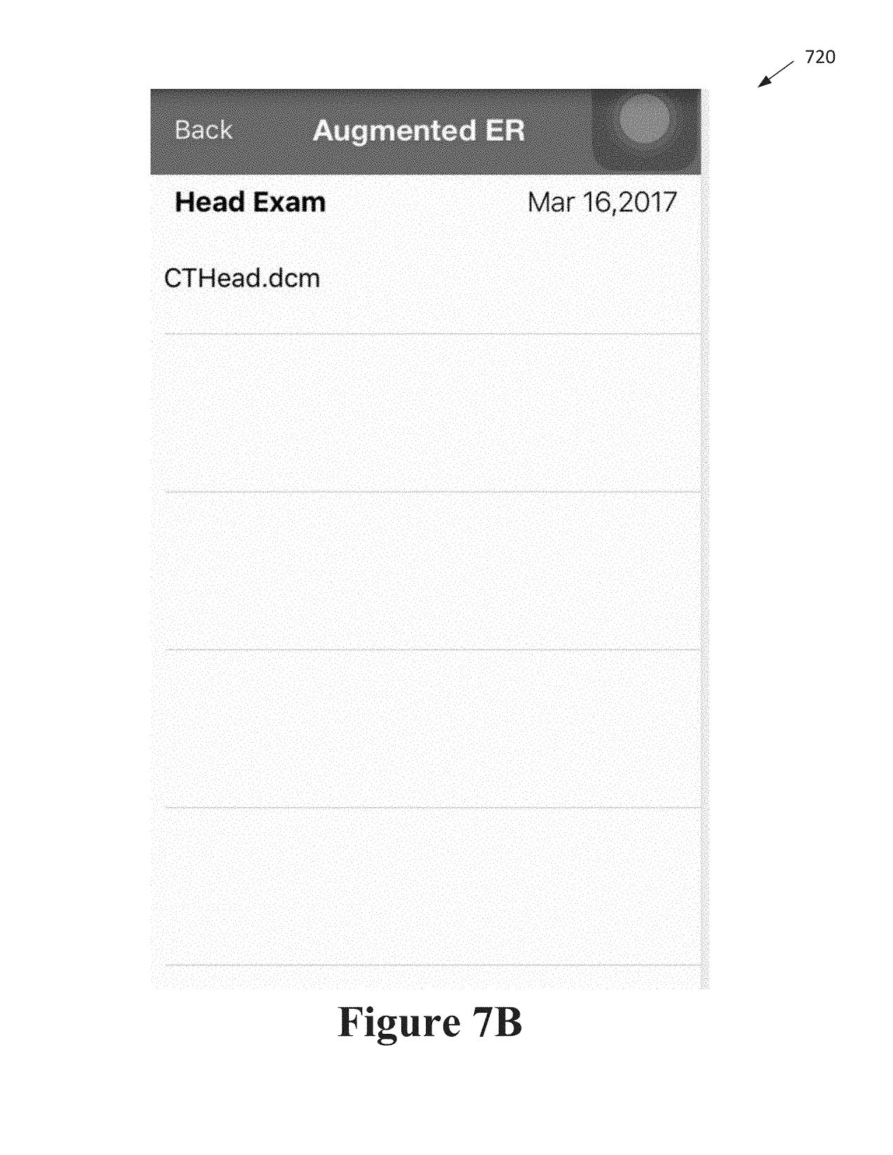

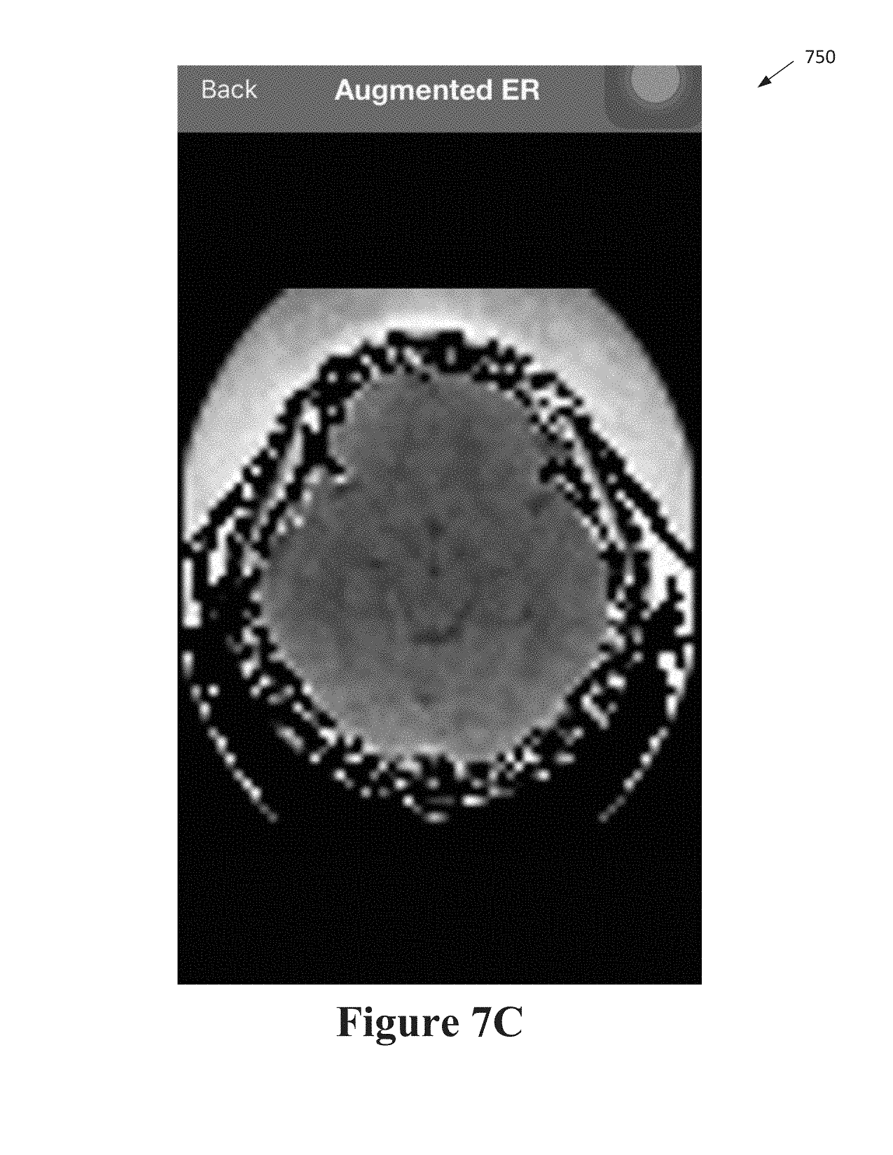

Returning to the Home Screen 600 and flow diagram 300, the user can also select the Augment Imaging 606 operation, step 320, from the Home Screen. Augmented imaging 606 displays medical images such as X-ray, MRI and CT scans. In contrast, Augmented EMR 604 is a combination of Imaging, Consultation data and LabReport as shown in FIG. 9B. In response to selecting Augment Imaging 606, the user is presented with the Augment Imaging screen 700 shown in FIG. 7A. The Augment Imaging screen 700 has a timeline selection 702 and an image display area 704. The image display area 704 includes the patient image 706 and annotations 708A, 708B, and 708C (collectively 708). The image displayed in image display area 704 may be a live video image, or a live picture of the patient, provided by the camera connected to the mobile application 200. That image may be automatically displayed on the Augment Imaging screen 700.

The Augment Imaging screen 700 may include one or more annotations 708. The annotations 708 are added to the image 706 based on the patient's medical records, and especially medical events. For ease of reference, the term “medical event” is used here to refer to injuries, illnesses, complaints, laboratory tests/results, reports, EMR encounters, or other medical related information. For example, if the patient has had a prior nose injury, then an annotation 708C may be added for the nose. When the patient image 706 is presented, those annotations 708 may be presented. As discussed above, the mobile application 200 may recognize various body parts captured in the actual patient image 706 to determine where annotations should be positioned on the image. For example, it determines where the patient's left eye is located, and adds an annotation “Left Eye” at the location of the patient's left eye, to indicate a prior eye injury.

The mobile application 200 identifies the body part that appears in the image 706 (e.g. eyes, nose, mouth, face), and adds the various annotations 708 to the image at the appropriate locations. The detection may be performed by the by the open CV parser 206(f) using a trained machine learning body parts model. As discussed above, the OpenCV trainer module 214 may be used to train the machine learning body parts model. The open CV parser 206(f) provides the coordinate in frame as (x,y) where a particular recognized body part is available in the frame. When the presentation layer 202 is provided with the image frame and the coordinate of the body part feature, the presentation layer 202 adds the annotation at that specific coordinate in image frame.

Annotations themselves can provide some indication of the medical event that was previously entered by the healthcare user when the record was created. For example, if the patient previously had an X-Ray taken of the mouth the annotation could read “Mouth; x-ray”. In addition, the annotation can indicate if the patient has several medical events at a same body part. For example, the annotation can say “Mouth; 10 events” or “Mouth; 10 injuries.”

The user can select (such as by clicking) on any of the displayed annotations 708 to view more detailed information about the prior medical event for the patient. The system may then display medical information related to that selected body part and medical event on a new screen. For example, the mobile application 200 can display images (pictures), laboratory results, reports, EMR encounters, etc., from a prior medical event. FIG. 7C is an example showing the CT of a patient's head 750.

The annotations 708 displayed may be associated to a period of time that the user selects in the timeline 702. When the user selects an annotation 708, the mobile application 200 retrieves medical information based on the selected period of time from the timeline 702. As shown, the timeline 702 includes several time periods, such as 3 months, 6 months, 1 year and 2 years. According to certain aspects, 3 months may be the default selection. The user may select all time periods to see all medical events for that patient from any time period. If the user selects “3 months,” the mobile application 200 will display only those annotations 708 and associated medical events that occurred during the last 3 months. By presenting the medical information in this visual manner, the healthcare professional may be able to quickly see all of the patient's medical issues at one time.

The Augment Imaging operation 320 also enables the user to enter a new patient medical event and/or edit patient records. The user can use a prior image or take a new picture of the injury for which the patient is currently seeking treatment and the system annotates that picture with the appropriate annotations. The user can then select a location (either annotated or unannotated) on the image where a new medical event has occurred at step 324. If the area is unannotated (i.e., a new body part for which there is no prior medical event for this particular patient), then the mobile application 200 can determine the appropriate annotation for that body part (e.g., cheek, right eye, etc.). The mobile application 200 then enables the user to select that newly-created annotation to enter specific information about that injury, as well as to add images, laboratory results, reports, EMR encounters, step 326.

The augment imaging operation 320 is handled by the imaging module 204(e). The information sent to 210 may be associated with and include patient identification information.

Selecting Augment Imaging 606 from FIG. 6, the presentation layer 202 displays the screen shown, for example, in FIG. 7A. The image is annotated and when the user selects an annotated icon, the presentation layer 202 passes the information to the Imaging Module 204(e). The Imaging Module 204(e) contains information on Augmented Imaging in an organized manner as fed by the HL7 Parser 206(e). The Imaging Module 204(e) responds with the information for the requested body part and the presentation layer 202 displays information for the requested body part 720 on the screen, such as shown for example in FIG. 7B. Once an entry from the screen in FIG. 7B is selected, the presentation layer 202 requests the information for that selected entry from the Imaging Module 204(e). The Imaging Module 204(e) responds with the information, which is then displayed as shown in FIG. 7C. According to certain aspects, imaging data may be received as digital imaging and communications in medicine (DICOM) data, which may be a combination of the images (can be single or multiple) along with patient details like name and ID. According to certain aspects the Augment Lab functionality may return the lab reports while Augment EMR functionality is a combination of Imaging, Lab and Consultation data. The Augment Lab 608 displays the laboratory reports for the patient with DICOM images and x-rays.

The Augment Lab 608 operation, step 330, may be handled by the Reports module 204(c) (FIG. 2) of the mobile application 200, as discussed above. On selecting Augment Lab 608, the presentation layer 202 sends information to the Reports Module 204(c). The Reports Module 204(c) may request information from the Lab Report Parser 206(c), which may obtain and parse lab reports stored in storage 207. The Lab Report Parser 206(c) responds with the required information and the presentation layer 202 displays that information, such as for example by the screen shown in FIG. 9C.

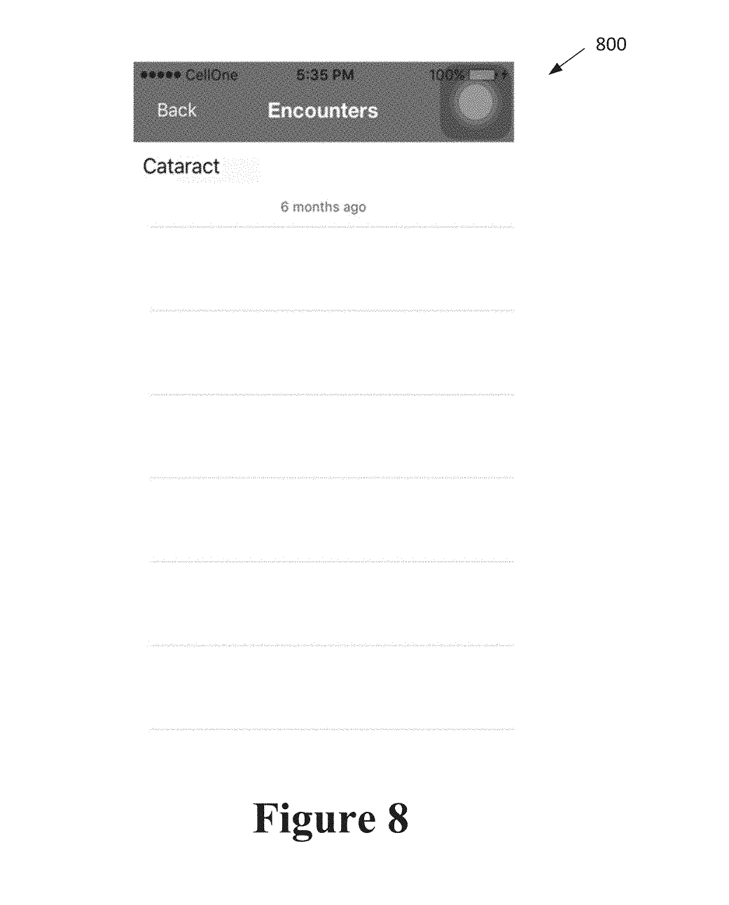

The user can also select the Fetch Encounters 610 operation, step 340, from the Home Screen. In response, the user is presented with the Encounters screen 800 shown in FIG. 8. The Encounters screen 800 displays appointments of the patient with healthcare workers, including previous appointments and upcoming appointments. Selecting a particular appointment may display details of the appointment, such as a date/time of the appointment, the medical professional the appointment is with, etc. FIG. 8 can show, for example, an appointment detail displayed with the disease and the time period since the appointment. This screen is displayed when the user selects Encounters 610 (FIG. 6) or Consultation Data (FIG. 9B).

The Fetch Encounters 610 operation is handled by the Encounters module 204(d) (FIG. 2) of the mobile application 200. On selecting the Fetch Encounters 610 from FIG. 6, the presentation layer 202 requests consultation data from the Encounter Module 204(d). The Encounter Module 204(d) receives information from the Encounter Parser 206(d). The Encounter Module 204(d) replies with the information it has and the presentation layer 202 displays that information, such as for example in the screen shown in FIG. 8.

The mobile application 200 can download and temporarily store all available medical information for the patient from the healthcare facility server 104 during the initial login, steps 304, 306, subject to any storage size constraints set on the application by, for example, the portable device. Alternatively, the mobile application 200 can communicate back and forth to retrieve and display only the information which the user has selected at any particular time. So for example, referring to FIG. 7A, the mobile application 200 can initially only retrieve information about prior injuries for a patient to display the annotations 708, and then subsequently retrieve that information selected by the user. If the user selects “nose”, then the mobile application 200 will request that specific information from the healthcare facility server 104 and display it to the user, without requesting or displaying information related to the other annotated features such as face, eyes, and mouth.

As illustrated by FIGS. 7, 8, and 10, the invention presents all the relevant information to the user in a simple and uncluttered manner. The user can then drill down to learn more specific information by selecting one of the annotations. Thus, the user can quickly and readily see all medical events for a patient at one time and learn more about any particular medical event as needed and ignore unrelated medical events. For example, if a patient comes into an emergency room with a bloody nose, the user can view only those medical events for the patient's nose, such as prior x-rays, pictures of past bloody noses, or the like. By selecting the nose, the user also bypasses all other medical information that is irrelevant to the current injury, such as a broken leg or skin cancer on the patient's arm. That also enables the mobile application 200 and healthcare facility server 104 to operate more quickly, as those components only need to provide information on the specific medical event at hand and not the totality of the patient's medical history. In addition, an unconscious patient in an ICU can be identified using his fingerprint. The patient gets the treatment faster as the doctor/physician need not wait for the details of the patient in paper which can take about 40-60 minutes, if not longer.

The system and method of the present invention include operation by one or more processing components or devices, including the mobile application 200 (and the various components, modules 204, parsers 206, and presentation layer 202), hand scan server 102, and healthcare facility server 104. It is noted that the processing device can be any suitable device, such as a computer, server, mainframe, processor, microprocessor, PC, tablet, smartphone, or the like. Thus, for example, the hand scan server 102 and/or the healthcare facility server 104 can be mainframe servers depending on the Handscan vendors and Hospitals, and a trainer module to train the system to identify the body parts, applications installed in tablets and phones and fingerprint scanners supporting mobile phones.

The processing devices can be used in combination with other suitable components, such as a display device (monitor, LED screen, digital screen, etc.), memory or storage device, input device (touchscreen, keyboard, pointing device such as a mouse), wireless module (for RF, Bluetooth, infrared, WiFi, etc.). The information may be stored on a computer hard drive, on a CD ROM disk or on any other appropriate data storage device or medium, which can be located at or in communication with the processing device. For example the information can be stored at the HIS 212, hand scan server 102 and within the application on the mobile application 200. The entire process is conducted automatically by the processing device, and without any manual interaction. Accordingly, unless indicated otherwise the process can occur substantially in real-time without any delays or manual action.

The operation of the processing device(s) is implemented by computer software that permits the accessing of data from an electronic information source. The software and the information in accordance with the invention may be within a single, free-standing computer or it may be in a central computer networked to a group of other computers or other electronic devices. The information may be stored on a computer hard drive, on a CD ROM disk or on any other appropriate data storage device.

Thus as used herein, the computing system or processing device includes a single electronic computing device that includes, but is not limited to a single computer, virtual machine, virtual container, host, server, laptop, and/or portable device or to a plurality of electronic computing devices working together to perform the function described as being performed on or by the computing system. And a medium includes one or more non-transitory physical media that together store the contents described as being stored thereon. Embodiments may include non-volatile secondary storage, read-only memory (ROM), and/or random-access memory (RAM). And an application includes one or more computing modules, programs, processes, workloads, threads and/or a set of computing instructions executed by a computing system. Example embodiments of an application include software modules, software objects, software instances and/or other types of executable code.

The foregoing description and drawings should be considered as illustrative only of the principles of the invention. The invention may be configured in a variety of manners and is not intended to be limited by the preferred embodiment. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

1. A patient healthcare information management system, comprising:
a hand scan server having a storage device configured to store a plurality of fingerprint data, each of the plurality of fingerprint data associated with unique patient identifying information for a respective one of a plurality of patients, and each of the plurality of fingerprint data identifying a respective one of the plurality of patients;
a fingerprint scanner configured to obtain fingerprint data of an examination patient being examined;
a processing device configured to receive the examination patient fingerprint data from the fingerprint scanner, forward the examination patient fingerprint data to said hand scan server, and in response receive the unique patient identifying information associated with the examination patient fingerprint data from the hand scan server.

2. The system of claim 1, wherein the patient identifying information comprises a patient ID or social security number.

3. The system of claim 1, wherein the processing device comprises a smartphone.

4. The system of claim 1, wherein the unique patient identifying information is associated with patient healthcare information.

5. The system of claim 4, wherein the processing device is further configured to retrieve the patient healthcare information for the examination patient based on the unique patient identifying information.

6. The system of claim 1, wherein the processing device is further configured to forward the unique patient identification information for the examination patient to a healthcare server and in response receiving patient healthcare information for the examination patient from the healthcare server.

7. The system of claim 6, wherein the processing device is further configured to display, on a display device, patient images with one or more annotations indicating patient healthcare information for the examination patient.

8. The system of claim 7, wherein the patient healthcare information for the examination patient is received from the healthcare server.

9. The system of claim 8, wherein the patient healthcare information for the examination patient is associated with one or more physical locations of the patient, and the one or more annotations is displayed at one or more image locations of the patient images corresponding to each of the physical locations.

10. The system of claim 9, wherein the processing device is further configured to determine, based on the patient healthcare information, the one or more image locations of the patient images to display the one or more annotations.

11. The system of claim 10, wherein the patient images are received from an imaging capture device.

12. The system of claim 11, wherein the imaging capture device comprises a camera.

13. A patient healthcare information management system, comprising:
a display device for displaying a patient image of a patient; and
a processing device configured to display on the display device, one or more annotations indicating patient healthcare information for the patient, wherein the patient healthcare information for the patient is associated with one or more physical locations of the patient, and said one or more annotations is displayed at one or more image locations of the patient image corresponding to each of the physical locations

14. The system of claim 13, wherein the patient healthcare information for the examination patient is received from a healthcare server.

15. The system of claim 13, wherein said processing device is further configured to determine, based on the patient healthcare information, the one or more image locations of the patient image to display the one or more annotations.

16. The system of claim 13, wherein the patient image is received from an imaging capture device.

17. The system of claim 16, wherein the imaging capture device comprises a camera.
